(19) United States
(12) Patent Application Publication
(10) Pub. No.: US 2024/0238579 A1
(43) Pub. Date: Jul. 18, 2024

(54) TRACHEOSTOMY TUBE COVER

(71) Applicant: Obiliz Technical Services LLC, Cheltenham, PA (US)

(72) Inventor: Obiora Chukwukadibia Ojukwu, Cheltenham, PA (US)

(21) Appl. No.: 18/424,008

(22) Filed: Jan. 26, 2024

Related U.S. Application Data
(63) Continuation of application No. 15/704,020, filed on Sep. 14, 2017.
(60) Provisional application No. 62/470,913, filed on Mar. 14, 2017.

(51) Int. Cl. A61M 39/20; A61M 16/04

(52) U.S. Cl. CPC A61M 39/20; A61M 16/04

(57) ABSTRACT

An example tracheal tube cover is described. The tracheal tube cover includes a tube section including an inner passage. The tube section is configured to detachably secure to an end of a tracheal tube. The tracheal tube cover includes a cap section positioned over and surrounding a portion of the tube section. The cap section includes an inner cavity. The tracheal tube cover includes one or more connectors coupling the cap section to the tube section. The inner passage of the tube section is in fluid communication with the inner cavity of the cap section.

### CROSS-REFERENCE TO RELATED APPLICATIONS

This application is a continuation of U.S. patent application Ser. No. 15/704,020, which was filed on Sep. 14, 2017, which claims the benefit of commonly assigned U.S. Provisional Patent Application No. 62/470,913, which was filed on Mar. 14, 2017. The entire content of the foregoing patent applications are incorporated herein by reference.

### BACKGROUND

Tracheal tubes are used in the medical industry to assist in patient breathing.

Particularly, a tracheal tube is generally inserted into the trachea to maintain an open airway for the purpose of ensuring an adequate exchange of oxygen and carbon dioxide. One end of the tracheal tube extends into the trachea and the opposing, open end protrudes from the throat of the patient such that oxygen and carbon dioxide can pass in and out of the tracheal tube as needed. During bathing of the patient, there is a risk of water entering the tracheal tube through the open end, resulting in aspiration and discomfort to the patient. In some instances, such water infiltration can lead to aspiration pneumonia.

### SUMMARY

Exemplary embodiments of the present disclosure provide a tracheal tube cover configured to attach over the open end of the tracheal tube to reduce or prevent water from entering the tracheal tube. The tracheal tube cover includes a tube section that secures to the tracheal tube and a cap section that is coupled to the tube section. Attached oxygen and carbon dioxide can travel between the tube section and the cap section to first enter a cavity formed in the cap section, and further travel from the cavity in the cap section through an opening in the tube section to enter the trachea. The tracheal tube cover therefore provides an efficient and effective means for occluding the open end of the tracheal tube during bathing of the patient.

In accordance with embodiments of the present disclosure, an exemplary tracheal tube cover is provided. The tracheal tube cover includes a tube section, a cap section, and one or more connectors. The tube section includes an inner passage, and is configured to detachably secure to an end of a tracheal tube. The cap section is positioned over and surrounds a portion of the tube section. The overlapping position of the cap section prevents airflow and/or water infiltration into the inner passage of the tube section. The cap section includes an inner cavity. The one or more connectors couple the cap section to the tube section. The inner passage of the tube section is in fluid communication with the inner cavity of the cap section to allow for airflow to travel between the tube section, the cap section, and the outside air.

In some embodiments, the tube section can define a substantially cylindrical configuration. In such embodiments, the inner passage of the tube section can define a substantially uniform diameter between proximal and distal ends of the tube section. The tube section can be concentrically disposed or coupled relative to the cap section. The cap section includes an opening at a proximal end and a closed inner surface at a distal end. In some embodiments, the inner cavity of the cap section can define a substantially uniform diameter between the opening and the closed inner surface.

The distal end of the tube section is spaced from the closed surface of the cap section to provide for unrestricted oxygen and/or carbon dioxide travel between the tube section and the cap section. A diameter of an outer surface of the tube section is dimensioned smaller than a diameter of an inner surface of the cap section. The tracheal tube cover includes a passage between the outer surface of the tube section and the inner surface of the cap section. The passage provides fluid communication between the outer surface of the tube section, the inner cavity of the cap section, and the inner passage of the tube section. The one or more connectors couple an inner surface of the cap section to an outer surface of the tube section.

In accordance with embodiments of the present disclosure, an exemplary tracheal tube system is provided. The tracheal tube system includes a tracheal tube and a tracheal tube cover. The tracheal tube includes an air intake line with an open end. The tracheal tube cover includes a tube section, a cap section, and one or more connectors. The tube section includes an inner passage, and is configured to detachably secure to an end of a tracheal tube. The cap section is positioned over and surrounds a portion of the tube section. The overlapping position of the cap section prevents water infiltration into the inner passage of the tube section. The cap section includes an inner cavity. The one or more connectors couple the cap section to the tube section. The inner passage of the tube section is in fluid communication with the inner cavity of the cap section to allow for oxygen and/or carbon dioxide to travel between the tube section, the cap section, and the outside air.

The open end of the air intake line of the tracheal tube is configured to be disposed externally of a human body such that the tracheal tube cover can be positioned over the open end. Particularly, the tube section is configured to receive the open end of the tracheal tube within the inner passage. In some embodiments, the inner passage of the tube section can define a substantially uniform diameter between proximal and distal ends of the tube section.

The tube section can be concentrically disposed relative to the cap section. The distal end of the tube section is spaced from the closed inner surface of the cap section. The diameter of an outer surface of the tube section is dimensioned smaller than a diameter of the inner surface of the cap section. The tracheal tube cover includes passage between the outer surface of the tube section and the inner surface of the cap section.

In accordance with embodiments of the present disclosure, an exemplary tracheal tube cover is provided. The tracheal tube cover includes a tube section, a cap section, and one or more connectors. The tube section includes an inner passage, and is configured to detachably secure to an end of a tracheal tube. The cap section is positioned over and surrounds a portion of the tube section. The cap section includes an inner cavity. The one or more connectors couple the cap section to the tube section. The diameter of an outer surface of the tube section is dimensioned smaller than the diameter of an inner surface of the cap section, forming a passage between the outer surface of the tube section and the inner surface of the cap section. The inner passage of the tube section is in fluid communication with the inner cavity of the cap section and the passage between the outer surface of the tube section and the inner surface of the cap section.

Other objects and features will become apparent from the following detailed description considered in conjunction with the accompanying drawings. It is to be understood, however, that the drawings are designed as an illustration only and not as a definition of the limits of the present disclosure.

### BRIEF DESCRIPTION OF THE DRAWINGS

To assist those of skill in the art in making and using the disclosed tracheal tube covers, below reference is made to the accompanying figures.

FIG. 1 is a perspective view of an exemplary tracheal tube cover of the present disclosure.

FIG. 2 is a side view of an exemplary tracheal tube cover of FIG. 1.

FIG. 3 is a perspective wireframe view of an exemplary tracheal tube cover of FIG. 1.

FIG. 4 is a side wireframe view of an exemplary tracheal tube cover of FIG. 1.

FIG. 5 is a cross-sectional view of an exemplary tracheal tube cover of FIG. 1 taken along line A-A of FIG. 6.

FIG. 6 is a top view of an exemplary tracheal tube cover of FIG. 1.

FIG. 7 is a bottom view of an exemplary tracheal tube cover of FIG. 1.

FIG. 8 is a side view of an exemplary tracheal tube cover positioned on a traditional tracheal tube.

### DETAILED DESCRIPTION

It should be understood that the scope of the present disclosure is not limited to embodiments having specific dimensions. Thus, any dimensions provided herein are merely for an exemplary purpose and are not intended to limit the invention to embodiments having particular dimensions.

The term “about” as used herein when referring to a measurable value such as a distance is meant to encompass variations of ±20%, ±10%, ±5%, ±1%, ±0.5%, ±0.4%, ±0.3%, ±0.2%, ±0.1%, ±0.09%, ±0.08%, ±0.07%, ±0.06%, ±0.05%, ±0.04%, ±0.03%, ±0.02% or ±0.01% from the specified value.

The term “connector” as used herein is defined as any structural component that directly or indirectly couples two structures. Such connector can be independently formed from the two structures or can be integrally formed with the two structures.

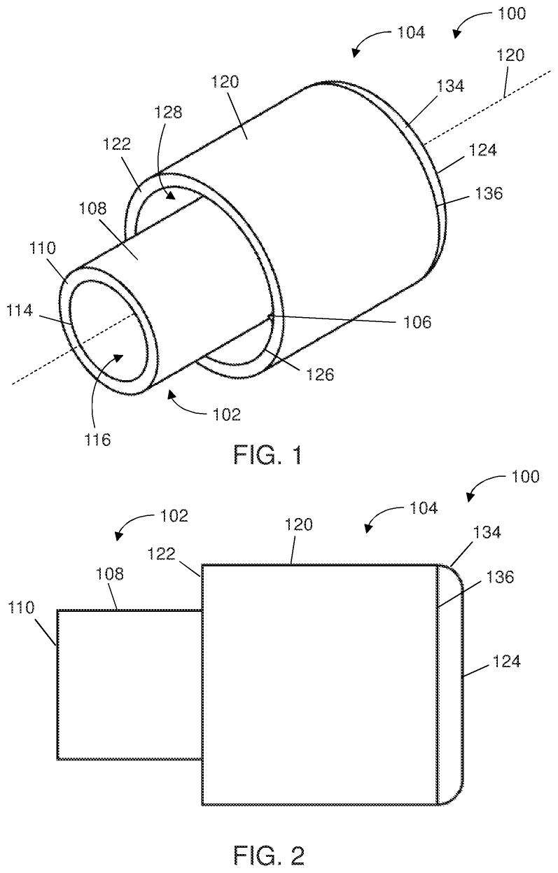

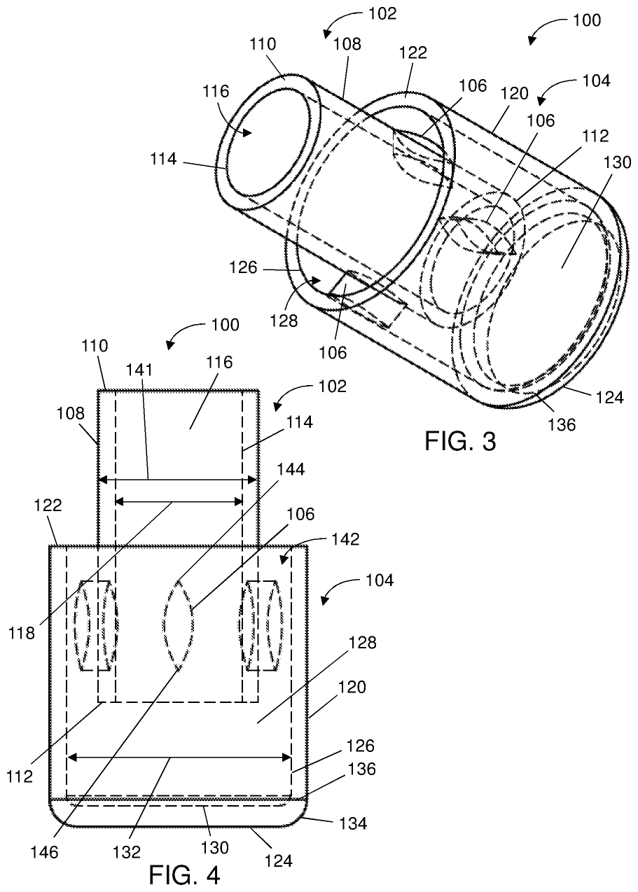

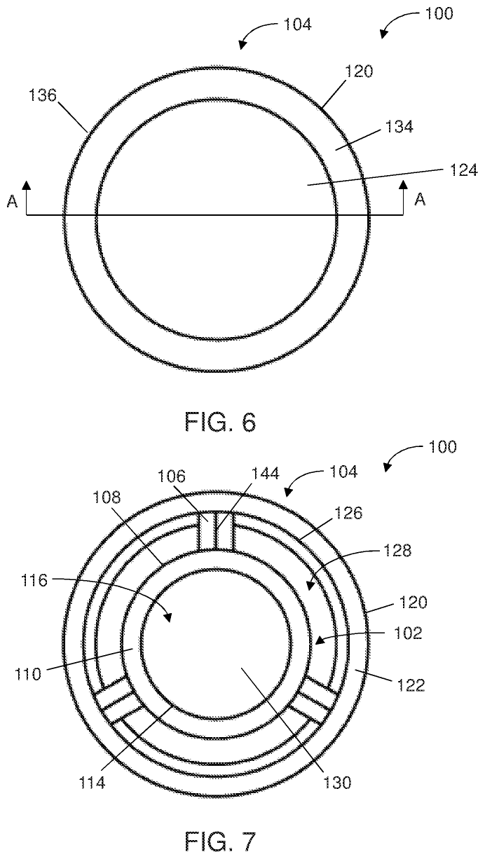

FIGS. 1 through 7 depict perspective, side, wireframe, cross-sectional, top and bottom views of an exemplary tracheal tube cover 100 (hereinafter “cover 100”). The cover 100 includes a tube section 102 and a cap section 104 coupled together by one or more connectors 106. In some embodiments, the cover 100 can be fabricated from a plastic material, e.g., polyvinyl chloride (PVC), or the like. In some embodiments, the cover 100 can be fabricated from the same material as the tracheal tube. In some embodiments, the cover 100 can be formed by, e.g., injection molding, or the like. The coupling between the tube and cap sections 102, 104 maintains the orientation and position of the tube section 102 relative to the cap section 104, thereby allowing airflow between the tube and cap sections 102, 104.

The tube section 102 defines a substantially cylindrical body with a rounded outer surface 108. The tube section 102 includes a proximal end 110 and a distal end 112 on opposing sides of the tube section 102. In some embodiments, the proximal and distal ends 110, 112 can both define substantially flat, circular surfaces. The tube section 102 includes an opening 114 formed in the body from the proximal end 110 to the distal end 112. The opening 114 forms an inner passage 116 extending through the tube section 102.

The opening 114 generally defines a substantially circular configuration with an inner diameter 118. In some embodiments, the diameter 118 can be dimensioned substantially uniformly between the proximal and distal ends 110, 112 (see, e.g., FIG. 4). The tube section 102 therefore defines a substantially symmetrical configuration relative to a central longitudinal axis 120 (see, e.g., FIG. 1). In some embodiments, the diameter 118 is about 0.61 inches. In some embodiments, the diameter 118 tapers between the proximal and distal ends 110, 112 (e.g., the diameter 118 can be greater at the proximal end 110 than the distal end 112; or the diameter 118 can be greater at the distal end 112 than the proximal end 110, or the like). In some embodiments, the diameter 118 is from about 0.4 to about 0.8 inches.

The cap section 104 defines a substantially cylindrical body with a rounded outer surface 120. In some embodiments, the outer diameter of the cap section 104 is about 1.24 inches. The cap section 104 includes a proximal end 122 and a distal end 124. The proximal end 122 can define a substantially flat, circular surface. The proximal end 122 includes an opening 126 that extends into an inner cavity 128 within the inner surfaces of the cap section 104. The opposing distal end 124 can define a substantially flat/planar, top outer surface of the cap section 104. The inner closed surface 130 at the distal end 124 of the cap section 104 can also define a substantially flat/planar surface.

The opening 126 can define a substantially circular configuration with an inner diameter 132. In some embodiments, the diameter 132 can be dimensioned substantially uniformly between the proximal and distal ends 122, 124 (see, e.g., FIG. 4). In such embodiments, the cap section 104 defines a substantially symmetrical configuration relative to the central longitudinal axis 120. In some embodiments, the diameter 132 can taper between the proximal and distal ends 122, 124 (e.g., the diameter 132 can be greater at the proximal end 122 than the distal end 124, the diameter 132 can be greater at the distal end 124 than the proximal end 122, or the like).

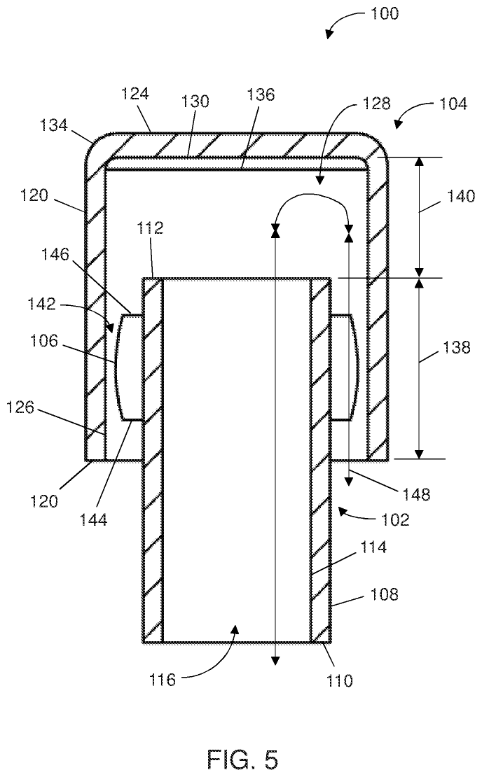

In some embodiments, cap section 104 can include a rounded or curved edge 134 connecting the flat distal end 124 to a connecting edge 136 of the body. From the connecting edge 136 to the proximal end 122, the outer diameter of the cap section 104 can be substantially uniform. The inner cavity 128 of the cap section 104 is configured and dimensioned to partially receive therein the tube section 102. Particularly, the distal end 112 of the tube section 102 is inserted partially into the inner cavity 128 by a partial length 138 such that a distance or space 140 is maintained between the distal end 112 of the tube section 102 and the inner closed surface 130 of the cap section 104 (see, e.g., FIG. 5). In some embodiments, the overall length of the tube section 102 (as measured along the central longitudinal axis 120) is about 1.5 inches, the overall length of the cap section 104 is about 1.35 inches, and the partial length 138 is about 0.75 inches (e.g., half the overall length of the tube section 102). In some embodiments, the overall length of the cap section 104 is from about 1 inch to about 2 inches.

The outer diameter 141 of the tube section 102 can be substantially uniform between the proximal and distal ends 110, 112, and is further dimensioned smaller than the inner diameter 132 of the cap section 104. The tube section 102 is therefore concentrically positioned within the inner cavity 128 of the cap section 104, with the tube section 102 and cap section 104 both aligned along the central longitudinal axis 120.

As noted above, the connectors 106 couple the outer surface 108 of the tube section 102 to the inner surface of the cap section 104, thereby maintaining the relative positions of the tube and cap sections 102, 104. Although shown as including three connectors 106 radially separated by about 60°, in some embodiments, one, two, three, four, five, six, or the like, connectors 106 can be used. The number of connectors 106 used is selected such that the passage 142 between the outer surface 108 of the tube section 102 and the inner surface of the cap section 104 remains substantially unobstructed to allow for oxygen and/or carbon dioxide therethrough.

Each of the connectors 106 includes a proximal end 144 and a distal end 146 (see, e.g., FIGS. 4 and 5). In some embodiments, as shown in FIG. 4, the sides of the connectors 106 can be curved or arcuate in form, and the proximal and distal ends 144, 146 can be pointed. Such pointed proximal and distal ends 144, 146 can ensure smoother travel of airflow through the passage 142. The connectors 106 therefore extend between the outer surface 108 of the tube section 102 and the inner surface of the cap section 104 to secure the tube section 104 relative to the cap section 104, while allowing for air flow 148 in both directions along the passage 142 (see, e.g., FIG. 5).

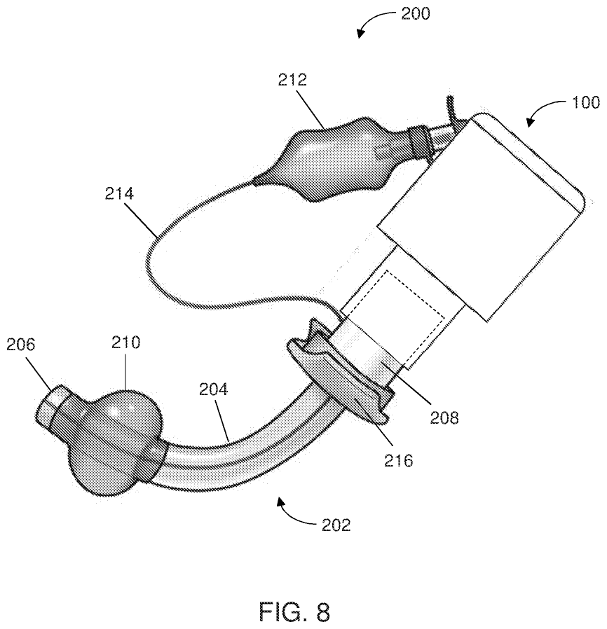

FIG. 8 shows a diagrammatic view of a tracheal tube system 200 (hereinafter “system 200”) that includes a tracheal tube 202 and the exemplary cover 100. Tracheal tubes 202 generally include an air intake tube or line 204 with one end 206 configured to be positioned within the trachea of a patient and an opposing open end 208 extending externally from the neck of the patient. In some embodiments, the open end 208 can include a connector 216 in the form of a flange extending outwardly from the air intake line 204. The tracheal tube 202 can also include a cuff 210 near the end 206, and a pilot balloon 212 connected to the air intake line 204 by a connecting line 214.

In operation, the air intake line 204 is inserted into the trachea of the patient up to the connector 216, with the open end 208 extending from the neck of the patient such that oxygen can be inhaled and carbon dioxide can be exhaled through the open end 208. During bathing of the patient, there is a risk of water entering the open end 208 and causing aspiration of the patient. The exemplary cover 100 reduces or prevents water infiltration into the open end 208 of the tracheal tube 202.

Particularly, as shown in FIG. 8, the proximal end 110 of the tube section 102 is configured to receive a portion of the open end 208 of the tracheal tube 202. Due to standard industry standards, the inner diameter 118 of the tube section 102 can be dimensioned to fit over the open end 208 and detachably secure to the open end 208 via a friction fit. With the cover 100 secured to the open end 208 of the tracheal tube 202, inhaling oxygen follows the air flow 148 pattern shown in FIG. 5 (e.g., oxygen travels into the cover 100 through the passage 142, into the inner cavity 128, into the inner passage 116, and into the air intake line 204). Exhaling carbon dioxide follows the opposing direction of air flow 148 pattern shown in FIG. 5 (e.g., carbon dioxide travels from the air intake line 204 into the inner passage 116, into the inner cavity 128, into the passage 142, and out of the cover 100).

The fluid communication between the air surrounding the cover 100 and the inner passages allows for unobstructed inhaling and exhaling of the patient. The depth at which tube section 102 is positioned within the inner cavity 128 ensures that sufficient coverage of the opening 114 in the tube section 102 is provided to prevent undesired water infiltration therethrough. The spacing between the distal end 112 of the tube section 102 and the inner surface 130 of the cap section 104 ensures that sufficient free space exists for air flow 148 to pass unobstructed. Particularly, implementation of the cover 100 over the tracheal tube 202 does not necessitate excess effort from the patient to breathe, and instead does not affect the breathing pattern of the patient. The cover 100 can therefore be conveniently and effectively placed over the tracheal tube 202 during bathing of the patient to prevent aspiration without obstructing other areas of the patient, and can be easily removed after the patient has been bathed.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

1. A tracheal tube cover, comprising:
a tube section comprising an outer surface and an inner passage, wherein the inner passage is configured to receive an end of a tracheal tube; and
a cap section comprising an inner cavity, wherein the inner cavity engages with one or more connectors radially positioned along the outer surface of the tube section to couple a distal end of the tube section to the cap section.

2. The tracheal tube cover of claim 1, wherein the tube section defines a cylindrical configuration.

3. The tracheal tube cover of claim 1, wherein the inner passage of the tube section defines a uniform diameter between proximal and distal ends of the tube section.

4. The tracheal tube cover of claim 1, wherein the tube section is concentrically disposed relative to the cap section.

5. The tracheal tube cover of claim 1, wherein the cap section comprises an opening at a proximal end and a closed surface at a distal end.

6. The tracheal tube cover of claim 5, wherein the inner cavity of the cap section defines a uniform diameter between the opening and the closed surface.

7. The tracheal tube cover of claim 5, further comprising a space based on coupling a partial length of the distal end of the tube section into the cap section.

8. The tracheal tube cover of claim 5, wherein a diameter of an outer surface of the tube section is dimensioned smaller than a diameter of an inner surface of the cap section.

9. The tracheal tube cover of claim 8, further comprising a passage between the outer surface of the tube section and the inner surface of the cap section unobstructed by the one or more connectors.

10. The tracheal tube cover of claim 9, wherein the passage provides fluid communication between the outer surface of the tube section, the inner cavity of the cap section, and the inner passage of the tube section.

11. The tracheal tube cover of claim 1, wherein a proximal end and a distal end of the one or more connectors are pointed.

12. The tracheal tube cover of claim 1, wherein at least one side of the one or more connectors is curved.

13. A tracheal tube system, comprising:
a tracheal tube comprising an air intake line with an open end; and
a tracheal tube cover, comprising:
a tube section comprising an outer surface and an inner passage, wherein the inner passage is configured to receive the open end of the air intake line of the tracheal tube; and
a cap section comprising an inner cavity, wherein the inner cavity engages with one or more connectors radially positioned along the outer surface of the tube section to couple a distal end of the tube section to the cap section.

14. The tracheal tube system of claim 13, wherein the open end of the air intake line of the tracheal tube is configured to be disposed externally of a human body.

15. The tracheal tube system of claim 13, wherein the tracheal tube cover further comprises a space based on coupling a partial length of the distal end of the tube section into the cap section.

16. The tracheal tube system of claim 13, wherein the inner passage of the tube section defines a uniform diameter between proximal and distal ends of the tube section.

17. The tracheal tube system of claim 13, wherein the tube section is concentrically disposed relative to the cap section.

18. The tracheal tube system of claim 13, wherein a distal end of the tube section is spaced from a closed inner surface of the cap section.

19. The tracheal tube system of claim 13, wherein the tracheal tube cover further comprises a passage between the outer surface of the tube section and the inner surface of the cap section unobstructed by the one or more connectors.

20. The tracheal tube system of claim 13, wherein a proximal end and a distal end of the one or more connectors are pointed and at least one side of the one or more connectors is curved.
